(19) United States
(12) Patent Application Publication
(10) Pub. No.: US 2006/0167119 A1
(43) Pub. Date: Jul. 27, 2006

(54) Fischer tropsch process

(76) Inventors: Stephen Anthony Leng, Ashford Middlesex (GB); Christopher Sharp, Beverley, East Yorkshire (GB)

Correspondence Address: NIXON & VANDERHYE, PC, 901 NORTH GLEBE ROAD, 11TH FLOOR, ARLINGTON, VA, 22203, US

(21) Appl. No.: 10/476,231

(22) PCT Filed: May 17, 2002
(86) PCT No.: PCT/GB02/02334

§ 371 Date: Mar. 12, 2004

(30) Foreign Application Priority Data
May 25, 2001 (GB) 0112792.7

(51) Int. Cl. C07C 27/26

(52) U.S. Cl. 518/726

(57) ABSTRACT

A process for the conversion of synthesis gas to product comprising liquid hydrocarbons wherein said process comprises contacting synthesis gas at an elevated temperature and pressure with a mixed particulate catalyst comprising a mixture of a particulate Fischer-Tropsch catalyst and a particulate hydrocracking and/or isomerisation catalyst.

The present invention relates to a process for the conversion of carbon monoxide and hydrogen (synthesis gas) to hydrocarbon products in the presence of a particulate catalyst.

In the Fischer-Tropsch reaction synthesis gas is reacted in the presence of a heterogeneous catalyst to give a hydrocarbon mixture having a relatively broad molecular weight distribution. This product comprises predominantly straight chain saturated hydrocarbons which typically have a chain length of more than 5 carbon atoms. However, in order to obtain the desired product distribution, high molecular weight waxes are generally produced in the Fischer-Tropsch synthesis reaction. These waxes may solidify which necessitates the use of heated pipelines.

It has now been found that the Fischer-Tropsch synthesis reaction can be combined with a hydrocracking and/or an isomerisation reaction.

The combined process provides a significant reduction in cost over a conventional two stage Fischer-Tropsch synthesis process and hydrocracking and/or isomerisation process where separate reactors are employed for the first and second stages and also eliminates the required heated pipelines for e.g. transporting the product of the conventional Fischer-Tropsch process to a hydrocracking and/or isomerisation stage.

Accordingly the present invention provides a process for the conversion of synthesis gas to a product comprising liquid hydrocarbons wherein said process comprises contacting synthesis gas at an elevated temperature and pressure with a mixed particulate catalyst comprising a mixture of a particulate Fischer-Tropsch catalyst and a particulate hydrocracking and/or an isomerisation catalyst.

Preferably the mixed particulate catalyst comprises a particulate Fischer-Tropsch catalyst and a particulate hydrocracking catalyst.

The mixed particulate catalyst may be located in a fixed or fluidized bed but preferably the process employs a slurry reactor e.g. a slurry bubble column in which the mixed particulate catalyst is primarily distributed and suspended in the slurry by the energy imparted from the synthesis gas rising from the gas distribution means at the bottom of the slurry bubble column as described in, for example, U.S. Pat. No. 5,252,613.

The mixed particulate catalyst may also be used in a reactor comprising at least one high shear mixing zone and a reactor vessel such as the reactor system described in WO 0138269 (PCT patent application number GB 0004444) which is herein incorporated by reference.

Accordingly, in a preferred embodiment of the invention the process comprises contacting synthesis gas at an elevated temperature and pressure with the mixed particulate catalyst comprising a particulate Fischer-Tropsch catalyst and a particulate hydrocracking and/or isomerisation catalyst suspended in a liquid medium in a reactor system comprising at least one high shear mixing zone and a reactor vessel wherein the process comprises:

a) passing the suspension through the high shear mixing zone(s) where the synthesis gas is mixed with the suspension;

b) discharging a mixture comprising the synthesis gas and the suspension from the high shear mixing zone(s) into the reactor vessel; and

c) converting the synthesis gas to liquid hydrocarbons in the reactor vessel to form a product suspension comprising the mixed particulate catalyst suspended in the liquid medium and liquid hydrocarbon products.

In order to simplify the process of the preferred embodiment it is preferred that the liquid medium is a liquid hydrocarbon.

Preferably the product suspension is, at least in part, recycled to the high shear mixing zone(s), as described in WO 0138269 (PCT patent application number GB 0004444). Preferably, a gaseous recycle stream comprising unconverted synthesis gas is withdrawn, either directly or indirectly, from the reactor vessel and is, at least in part, recycled to the high shear mixing zone(s), also as described in WO 0138269 (PCT patent application number GB 0004444).

The reactor vessel may be a tank reactor or a tubular loop reactor. The high shear mixing zone(s) may be part of the reactor system inside or outside the reactor vessel, for example, the high shear mixing zone(s) may project through the walls of the reactor vessel such that the high shear mixing zone(s) discharges its contents into the reactor vessel. Preferably, the reactor system comprises up to 250 high shear mixing zones, more preferably less than 100, most preferably less than 50, for example 10 to 50 high shear mixing zones. Preferably, the high shear mixing zones discharge into or are located within a single reactor vessel as described in WO 0138269 (PCT patent application number GB 0004444). It is also envisaged that 2 or 3 such reactor systems may be employed in series.

Suitably, the volume of suspension present in the high shear mixing zone(s) is substantially less than the volume of suspension present in the reactor vessel, for example, less than 20%, preferably less than 10% of the volume of suspension present in the reactor vessel.

The high shear mixing zone(s) may comprise any device suitable for intensive mixing or dispersing of a gaseous stream in a suspension of solids in a liquid medium, for example, a rotor-stator device, an injector-mixing nozzle or a high shear pumping means, but is preferably an injector mixing nozzle(s). Suitably, the device is capable of breaking down the gaseous stream into gas bubbles and/or irregularly shaped gas voids.

The kinetic energy dissipation rate in the high shear mixing zone(s) is suitably, at least 0.5 kW/m³ relative to the total volume of suspension present in the system, preferably in the range 0.5 to 25 kW/m³, more preferably 0.5 to 10 kW/m³, most preferably 0.5 to 5 kW/m³, and in particular, 0.5 to 2.5 kW/m³ relative to the total volume of suspension present in the system.

Where the high shear mixing zone(s) comprise an injector mixing nozzle the injector-mixing nozzle(s) can advantageously be executed as a venturi tube (c.f. “Chemical Engineers' Handbook” by J. H. Perry, 3^{rd} edition (1953), p. 1285, FIG. 61), preferably an injector mixer (c.f. “Chemical Engineers' Handbook” by J H Perry, 3^{rd} edition (1953), p 1203, FIG. 2 and “Chemical Engineers' Handbook” by R H Perry and C H Chilton 5^{th} edition (1973) p 6-15, FIG. 6-31) or most preferably as a liquid-jet ejector (c.f. “Unit Operations” by G G Brown et al, 4^{th} edition (1953), p. 194, FIG. 210). The injector mixing nozzle(s) may also be executed as a venturi plate positioned within an open ended conduit which discharges the mixture of synthesis gas and suspension into a tank reactor. Alternatively the venturi plate may be positioned within a tubular loop reactor. Suitably, synthesis gas is introduced into the open-ended conduit or tubular loop reactor downstream of the venturi plate. The injector-mixing nozzle(s) may also be executed as “gas blast” or “gas assist” nozzles where gas expansion is used to drive the nozzle (c.f. “Atomisation and Sprays” by Arthur H Lefebvre, Hemisphere Publishing Corporation, 1989). Where the injector-mixing nozzle(s) is executed as a “gas blast” or “gas assist” nozzle, the suspension of catalyst is fed to the nozzle at a sufficiently high pressure to allow the suspension to pass through the nozzle while the gaseous reactant stream is fed to the nozzle at a sufficiently high pressure to achieve high shear mixing within the nozzle.

The high shear mixing zone(s) may also comprise a high shear pumping means, for example, a paddle or propeller having high shear blades positioned within an open ended pipe which discharges the mixture of synthesis gas and suspension into the reactor vessel. Preferably, the high shear pumping means is located at or near the open end of the pipe, for example, within 1 metre preferably within 0.5 metres of the open end of the pipe. Alternatively, the high shear pumping means may be positioned within a tubular loop reactor vessel. Synthesis gas may be injected into the pipe or tubular loop reactor vessel, for example, via a sparger, located immediately upstream or downstream, preferably upstream of the high shear pumping means, for example, preferably, within 1 metre, preferably within 0.5 metre of the high shear pumping means. Without wishing to be bound by any theory, the injected synthesis gas is broken down into gas bubbles and/or irregularly shaped gas voids by the fluid shear imparted to the suspension by the high shear pumping means.

Where the injector mixing nozzle(s) is executed as a venturi nozzle(s) (either a conventional venturi nozzle or as a venturi plate), the pressure drop of the suspension over the venturi nozzle(s) is typically in the range of from 1 to 40 bar, preferably 2 to 15 bar, more preferably 3 to 7 bar, most preferably 3 to 4 bar. Preferably, the ratio of the volume of gas (Q_{g}) to the volume of liquid (Q₁) passing through the venturi nozzle(s) is in the range 0.5:1 to 10:1, more preferably 1:1 to 5:1, most preferably 1:1 to 2.5:1, for example, 1:1 to 1.5:1 (where the ratio of the volume of gas (Q_{g}) to the volume of liquid (Q₁) is determined at the desired reaction temperature and pressure).

Where the injector mixing nozzle(s) is executed as a gas blast or gas assist nozzle(s), the pressure drop of gas over the nozzle(s) is preferably in the range 3 to 100 bar and the pressure drop of suspension over the nozzle(s) is preferably in the range of from 1 to 40 bar, preferably 4 to 15, most preferably 4 to 7. Preferably, the ratio of the volume of gas (Q_{g}) to the volume of liquid (Q₁) passing through the gas blast or gas assist nozzle(s) is in the range 0.5:1 to 50:1, preferably 1:1 to 10:1 (where the ratio of the volume of gas (Q_{g}) to the volume of liquid (Q₁) is determined at the desired reaction temperature and pressure).

Suitably, the shearing forces exerted on the suspension in the high shear mixing zone(s) are sufficiently high that the synthesis gas is broken down into gas bubbles having diameters in the range of from 1 μm to 10 mm, preferably from 30 μm to 3000 μm, more preferably from 30 μm to 300 μm.

Without wishing to be bound by any theory, it is believed that the irregularly shaped gas voids are transient in that they are coalescing and fragmenting on a time scale of up to 500 ms, for example, over a 10 to 50 ms time scale. The irregularly shaped gas voids have a wide size distribution with smaller gas voids having an average diameter of 1 to 2 mm and larger gas voids having an average diameter of 10 to 15 mm.

The high shear mixing zone(s) can be placed at any position on the walls of the reactor vessel (for example, at the top, bottom or side walls of a tank reactor). Where the reactor vessel is a tank reactor the suspension is preferably withdrawn from the reactor vessel and is at least in part recycled to a high shear mixing zone(s) through an external conduit having a first end in communication with an outlet for suspension in the reactor vessel and a second end in communication with an inlet of the high shear mixing zone. The suspension may be recycled to the high shear mixing zone(s) via a pumping means, for example, a slurry pump, positioned in the external conduit. Owing to the exothermic nature of the Fischer-Tropsch synthesis reaction, the suspension recycle stream is preferably cooled by means of a heat exchanger positioned on the external conduit (external heat exchanger). Additional cooling may be provided by means of an internal heat exchanger comprising cooling coils, tubes or plates positioned within the suspension in the tank reactor.

Suitably, the ratio of the volume of the external conduit (excluding the volume of any external heat exchanger) to the volume of the tank reactor is in the range of 0.005:1 to 0.2:1.

Where the reactor vessel is a tubular loop reactor, a single high shear mixing zone, in particular an injector-mixing nozzle may discharge the mixture comprising synthesis gas and the suspension into the tubular loop reactor. Alternatively, a series of injector-mixing nozzles may be arranged around the tubular loop reactor. If necessary, suspension may be circulated around the tubular loop reactor via at least one mechanical pumping means e.g. a paddle or propeller. An external heat exchanger may be disposed along at least part of the tubular loop reactor, preferably along substantially the entire length of the tubular loop reactor thereby providing temperature control. It is also envisaged that an internal heat exchanger, for example cooling coils, tubes or plates may-be located in at least part of the tubular loop reactor.

Preferably the Fischer-Tropsch reactor system of the preferred embodiment is operated with a gas hourly space velocity (GHSV) in the range of 100 to 40000 h⁻¹, more preferably 1000 to 30000 h⁻¹, most preferably 2000 to 15000, for example 4000 to 10000 h⁻¹ at normal temperature and pressure (NTP) based on the feed volume of synthesis gas at NTP.

Usually the suspension discharged into the reactor vessel from the high shear mixing zone(s) comprises less than 40% wt of mixed catalyst particles, more preferably 10 to 30% wt of mixed catalyst particles, most preferably 10 to 20% wt of mixed catalyst particles.

The process of the invention reaction is preferably carried out at a temperature of 180-280° C., more preferably 190-240° C.

The process of the invention is preferably carried out at a pressure of 5-50 bar, more preferably 15-35 bar, generally 20-30 bar.

The synthesis gas may be prepared using any of the processes known in the art including partial oxidation of hydrocarbons, steam reforming, gas heated reforming, microchannel reforming (as described in, for example, U.S. Pat. No. 6,284,217 which is herein incorporated by reference), plasma reforming, autothermal reforming and any combination thereof. A discussion of these synthesis gas production technologies is provided in “Hydrocarbon Processing” V78, N.4, 87-90, 92-93 (April 1999) and “Petrole et Techniques”, N. 415, 86-93 (July-August 1998). It is also envisaged that the synthesis gas may be obtained by catalytic partial oxidation of hydrocarbons in a microstructured reactor as exemplified in “IMRET 3: Proceedings of the Third International Conference on Microreaction Technology”, Editor W Ehrfeld, Springer Verlag, 1999, pages 187-196. Alternatively, the synthesis gas may be obtained by short contact time catalytic partial oxidation of hydrocarbonaceous feedstocks as described in EP 0303438. Preferably, the synthesis gas is obtained via a “Compact Reformer” process as described in “Hydrocarbon Engineering”, 2000, 5, (5), 67-69; “Hydrocarbon Processing”, 79/9, 34 (September 2000); “Today's Refinery”, 15/8, 9 (August 2000); WO 99102254; and WO 200023689.

Preferably, the ratio of hydrogen to carbon monoxide in the synthesis gas is in the range of 20:1 to 1:1 by volume and especially in the range of 5:1 to 1:1 by volume e.g. 2:1 by volume.

Preferably, the hydrocarbons produced by contact of the synthesis gas with the Fischer-Tropsch catalyst comprise a mixture of hydrocarbons having a chain length of greater than 5 carbon atoms. Suitably, the hydrocarbons comprise a mixture of hydrocarbons having chain lengths of from 5 to about 90 carbon atoms. Preferably, a major amount, for example, greater than 60% by weight, of the hydrocarbons have chain lengths of from 5 to 30 carbon atoms.

The hydrocarbons produced by contact of the synthesis gas with the Fischer-Tropsch catalyst and the hydrocracking and/or the isomerisation catalyst produces hydrocarbons of shorter chain length and/or an increased degree of branching than hydrocarbons produced in the absence of the hydrocracking and/or the isomerisation catalyst.

The final hydrocarbon product of the process of the present invention may comprise light gasoline with a TBP (True Boiling Point) range of 0-70° C., naphtha with a TBP of 70-140° C., kerosine with a TBP of 140-250° C., diesel fuel with a TBP of 250-350° C. TBP or lubricating basestock and speciality wax with a TBP above 350° C. Preferably the final hydrocarbon product is a light gasoline or a diesel fuel, especially a diesel fuel.

The catalytic composition employed in the process of the present invention comprises a combination of any catalyst known to be active in Fischer-Tropsch synthesis and any catalyst known to be active in the hydrocracking and/or isomerisation of hydrocarbons. The ratio of Fischer-Tropsch catalyst to hydrocracking catalyst is usually in the range of 25:1 to 1:10 preferably 20:1 to 1:1 and especially 15:1 to 5:1 e.g. 12:1 by weight. The ratio of Fischer-Tropsch catalyst to isomerisation catalyst is usually in the range of 25:1 to 1:10 preferably 20:1 to 1:1 and especially 15:1 to 5:1 e.g. 12:1 by weight.

Fischer-Tropsch catalysts usually comprise supported or unsupported Group VIII metals. Of these iron, cobalt and ruthenium are preferred, particularly iron and cobalt, most particularly cobalt.

A preferred catalyst is supported on an inorganic oxide, preferably a refractory inorganic oxide. Preferred supports include silica, alumina, silica-alumina, the Group IVB oxides, titania (primarily in the rutile form) and most preferably zinc oxide. The supports generally have a surface area of less than about 100 m²/g, suitably less than 50 m²/g, for example, less than 25 m²/g or about 5 m²/g.

The catalytic metal is present in catalytically active amounts usually about 1-100 wt %, the upper limit being attained in the case of metal based catalysts, preferably 2-40 wt %. Promoters may be added to the catalyst and are well known in the Fischer-Trospch catalyst art. Promoters can include ruthenium, platinum or palladium (when not the primary catalyst metal), aluminium, rhenium, hafnium, cerium, lanthanum and zirconium, and are usually present in amounts less than the primary catalytic metal (except for ruthenium which may be present in coequal amounts), but the promoter:metal ratio should be at least 1:10. Preferred promoters are rhenium and hafnium.

Hydrocracking catalysts usually comprise a metal selected from the group consisting of platinum, palladium, cobalt, molybdenum, nickel and tungsten supported on a support material such as alumina, silica-alumina or a zeolite. Preferably, the catalyst comprises either cobalt/molybdenum or platinum supported on alumina or platinum or palladium supported on a zeolite. The most suitable hydrocracking catalysts include catalysts supplied by Akzo Nobel, Criterion, Chevron, or UOP. A preferred catalyst is KF 1022™, a cobalt/molybdenum supported on silica alumina catalyst, supplied by Akzo Nobel.

Isomerisation catalysts are usually acidic in nature e.g. alumina, silica-alumina or a zeolite. Advantageously the isomerisation catalyst is a Friedel-Crafts acid which comprises a metal halide, especially a chloride or a bromide, of transition metals of Groups IIIA to IIIB of the Periodic Table (in F. A. Cotton & G. Wilkinson Advanced Inorganic Chemistry Publ. Interscience 1966) and elements of Groups IIIB-VB. Thus examples are chlorides of iron, zinc, titanium and zirconium, and chlorides and fluorides of boron, aluminium, antimony and arsenic. Preferred catalysts are boron trifluoride, ferric chloride and niobium and tantalum and antimony pentafluoride.

The hydrocracking catalysts may also be capable of acting as isomerisation catalysts in particular those wherein the metals are supported on alumina, silica-alumina or a zeolite, whilst the isomerisation catalyst may also exhibit some hydrocracking activity.

The isomerisation and/or hydrocracking catalyst generally has a surface area of less than about 450 m²/g, preferably less than 350 m²/g, more preferably less than 300 m²/g, for example, about 200 m²/g.

The mixed particulate catalyst may have an average particle size in the range 5 to 500 microns, preferably 5 to 100 microns, for example, in the range 5 to 40 microns. The average particle size of the Fischer-Tropsch catalyst may be the same or different to that of the hydrocracking and/or the isomerisation catalyst. Generally the average particle sizes of the Fischer-Tropsch catalyst and hydrocracking and/or isomerisation catalyst are substantially the same when used in a fixed or fluidized bed reactor i.e. unimodal particle size distribution. When slurry reactors are used and especially when tank or tubular loop reactors are employed (as herein described above) the Fischer-Tropsch catalyst may have a different average particle size to that of the hydrocracking and/or the isomerisation catalyst i.e. bimodal particle size distribution and when a Fischer-Tropsch catalyst, a hydrocracking catalyst and an isomerisation catalyst are employed all three may advantageously have a different average particle size i.e. trimodal particle size distribution.

The invention will now be described with reference to the following example.

### EXAMPLE 1

A 9 mm diameter tubular fixed bed reactor was loaded with 5 ml of a Fischer-Tropsch catalyst comprising 10% by weight of Co supported on ZnO and 5 ml of a hydrocracking catalyst KF 1022™ comprising cobalt/molybdenum supported on silica alumina catalyst.

Hydrogen was then passed to the reactor at a gas hourly space velocity (GHSV) of 1500 h⁻¹ and the reactor was heated at 2° C. min⁻¹ to 280° C. and then held at 280° C. for 4 hours. The reactor was then allowed to cool to room temperature.

Synthesis gas was passed to the reactor at a GHSV of 2000 h⁻¹. The synthesis gas contained 27% by weight CO, 54% by weight H₂ and 19% by weight N₂.

The reactor was then pressurised to 30 bar and the flow rate of synthesis gas was reduced to a GHSV of 1250 h⁻¹. The reactor temperature was raised at 2° C. min⁻¹ to 175° C. The temperature was increased until at least 60% CO conversion was achieved. The product gases were analysed and the results are shown in Table 1.

### COMPARATIVE EXAMPLE

The hydrocracking catalyst was removed and example 1 was repeated using 10 ml of the Fischer-Tropsch catalyst. The product gases were analysed and the results are shown also in Table 1.

TABLE 1

| | Fischer-Tropsch and | Fischer-Tropsch |
|---|---|---|
| | hydrocracking catalyst | catalyst. |
| Run Temperature ° C. | 209 | 213 |
| Pressure Bar | 30 | 30 |
| CO % Conversion | 62.6 | 62.6 |
| CO₂ % Selectivity | 1.9 | 1.6 |
| C₁-C₅ % Selectivity | 23.3 | 20.8 |
| C₅₊ % Selectivity | 74.8 | 77.6 |

It can bee seen from the above examples that the addition of the hydrocracking catalyst results in a lower selectivity to C₅₊ hydrocarbons and an increased selectivity to C₁-C₅ hydrocarbons.

Analysis of the Wax Products

The wax products resulting from the above examples were analysed. The alpha values and maximum carbon numbers for the resulting products were ascertained using the Schulz-Flory Distribution wherein
Wₙ=(1-Alpha)²nAlpha⁽ⁿ⁻¹⁾
n Carbon Number
Wn=Weight fraction of product with carbon number n

$$\begin{matrix}Alpha = Schulz Flory distribution factor \\ = Rate of Chain Propagation / Rate of Chain Propagation + \\ Rate of Termination\end{matrix}$$

The Alpha values were determined by plotting log (Wn/n) against n
log (Wₙ/n)=log [(1-Alpha)²/Alpha]+n log Alpha.

The results are shown in table 2.

TABLE 2

| | Fischer-Tropsch and | Fischer-Tropsch |
|---|---|---|
| | hydrocracking catalyst | catalyst |
| Alpha Value | 0.869 | 0.890 |
| Maximum Carbon number | 80 | 98 |

It can be seen that a lighter product is produced with the combination of the Fischer-Tropsch catalyst and hydrocracking catalyst than that produced when using the Fischer-Tropsch catalyst alone.

1. A process for the conversion of synthesis gas to a product comprising liquid hydrocarbons wherein said process comprises contacting synthesis gas at an elevated temperature and pressure with a mixed particulate catalyst comprising a mixture of a particulate Fischer-Tropsch catalyst and a particulate hydrocracking and/or isomerisation catalyst.

2. A process according to claim 1 wherein the process comprises contacting synthesis gas at elevated temperature and pressure with the mixed particulate catalyst comprising a particulate Fischer-Tropsch catalyst and a particulate hydrocracking and/or isomerisation catalyst suspended in a liquid medium in a reactor system comprising at least one high shear mixing zone and a reactor vessel wherein the process comprises:
a) passing the suspension through the high shear mixing zone(s) where the synthesis gas is mixed with the suspension;
b) discharging a mixture comprising the synthesis gas and the suspension from the high shear mixing zone(s) into the reactor vessel; and
c) converting the synthesis gas to liquid hydrocarbons in the reactor vessel to form a product suspension comprising the mixed particulate catalyst suspended in the liquid medium and liquid hydrocarbon products.

3. A process according to claim 2 wherein the reactor vessel is a tank reactor or a tubular loop reactor.

4. A process according to claim 2 wherein the high shear mixing zone(s) project through the walls of the reactor vessel such that the high shear mixing zone(s) discharges its contents into the reactor vessel or is located within the reactor vessel.

5. A process according to claim 2 wherein the reactor system comprises up to 250 high shear mixing zones.

6. A process according claim 2 wherein the high shear mixing zone(s) comprise an injector-mixing nozzle(s).

7. A process according to claim 6 wherein the injector mixing nozzle(s) is a venturi nozzle(s) or a gas blast nozzle(s).

8. A process according to claim 1 wherein the Fischer-Tropsch reaction is carried out at a temperature of 180-280° C. and at a pressure of 5-50 bar.

9. A process according to claim 1 wherein the ratio of hydrogen to carbon monoxide in the synthesis gas is in the range of 20:1 to 0.1:1 by volume.

10. A process according to claim 1 wherein the ratio of Fischer-Tropsch catalyst to hydrocracking and/or the isomerisation catalyst is the range of 25:1 to 1:10 by weight.

11. A process according to claim 1 wherein the mixed particulate catalyst comprises a mixture of a Fischer-Tropsh catalyst and a hydrocracking catalyst.

12. A process according to claim 11 wherein the hydrocracking catalyst is cobalt and molybdenum supported on silica-alumina.

13. A process according to claim 1 wherein the Fischer-Tropsch catalyst is cobalt supported on zinc oxide.
